(19) United States
(12) Patent Application Publication
(10) Pub. No.: US 2014/0066847 A1
(43) Pub. Date: Mar. 6, 2014

(54) FOOT IMPLANT AND METHOD FOR TREATING A FOOT

(71) Applicant: Dinusha Lalwani, Corona, CA (US)

(72) Inventor: Dinusha Lalwani, Corona, CA (US)

(73) Assignee: ALLERGAN, INC., Irvine, CA (US)

(21) Appl. No.: 13/601,152

(22) Filed: Aug. 31, 2012

(51) Int. Cl. A61F 2/00

(52) U.S. Cl. CPC A61F 2/0063
USPC 604/93.01; 606/151

(57) ABSTRACT

A silk mesh used for treating plantar fasciitis and a method of treating plantar fasciitis. The silk mesh optionally comprises a low dose corticosteroid. The method of treating plantar fasciitis comprises inserting a silk, biocompatible surgical mesh over the plantar aspect of the foot to treat plantar fasciitis.

The present invention relates to a treatment for a foot disease, ailment or condition. In particular the present invention relates to use of an implantable surgical mesh or fabric to treat plantar fasciitis.

### BACKGROUND

The fields of bioengineering, biomaterials and tissue engineering are providing new options to gradually restore native tissue and overcome injuries through the research and development of scaffolds, meshes, matrices and constructs which are often referred to as devices that initially support a disabled portion of the body, but eventually allow for the development and remodeling of the body's own biologically and mechanically functional tissue. Hence, surgical meshes and scaffolds have been used historically in a variety of applications to reinforce tissue or other structures where defects or weakness exist. However, certain medical, cosmetic and surgical applications present unique challenges with regard to tissue and structural repair.

For example, every year, hundreds of thousands of people sprain, tear, or rupture tissue, ligaments and tendons of the knee, elbow, ankle, foot, hand, shoulder, wrist and jaw. One such area is the plantar fascia which is a thick fibrous band of connective tissue originating on the bottom surface of the calcaneus (heel bone) and extending along the sole of the foot towards the heads of the metatarsal bones. The plantar fascia supports the arch of the foot and acts as a spring when the foot bears weight.

The plantar fascia can become damaged and create a painful and long lasting condition called plantar fasciitis. It has been reported that plantar fasciitis occurs in two million Americans a year and 10% of the population over a lifetime. Among non-athletic populations, plantar fasciitis is associated with a high body mass index. Most often the pain is felt under the heel of the foot and may also lead to decreased ankle dorsiflexion.

Plantar fasciitis is a musculoskeletal disorder primarily affecting the point where the ligament inserts into the bone. Although poorly understood, the development of plantar fasciitis is thought to have a mechanical origin. In particular, pes planus (flat) foot types and lower-limb biomechanics that result in a lowered medial longitudinal arch are thought to create excessive tensile strain within the fascia, producing microscopic tears and chronic inflammation, all of which may affect gait and contribute to gait related injuries. While there are several treatment modalities that exist for this condition, none have been effective over the long term use.

It is widely known that plantar fasciitis is a long lasting condition and that the plantar fascia has poor healing capabilities. Persistent pain and inflammation around the heel create a debilitating condition that is often felt upon waking As the plantar fascia is stretched and becomes more limber, the pain subsides. Oftentimes, however, the pain returns after exercise or long periods of standing.

Conventionally, the techniques for treating plantar fasciitis include medications, such as corticosteroids, and nonsteroidal anti-inflammatory drugs, such as ibuprofen. Corticosteroids may be delivered by applying a solution over the skin in a process called iontophoresis. A small, nonpainful electric current is applied to the solution to aid absorption of the corticosteroid into the skin. Direct injection of corticosteroids is also a common delivery method. However, while use of corticosteroids has been shown to be effective, such use can cause potential rupture of the plantar fascia after multiple injections. Some studies have found that a maximum of three corticosteroid injections can be given to a patient per year before potential weakening and rupture of the plantar fascia. In addition, the studies found less than 30 days of relief with injectable corticosteroids.

Various therapies are also employed for stretching and strengthening the plantar fascia and surrounding tissues. Physical therapy is a common treatment modality that includes exercises to stretch the plantar fascia and add stability to the surrounding muscles and tissues. Athletic taping may also be employed to support the plantar fascia, and/or night splints may be used to put the foot into a dorisflexion state while the patient sleeps in order to facilitate stretching. Other procedures may also be employed, such as directing sound waves or electrical pulses at the heel area to stimulate healing.

Surgery may also be performed to detach the plantar fascia from the heel bone, but this is typically seen as a last resort and often results in a near or complete collapse of the foot arch. Thus, there exists a need for a structure and method for treating plantar fasciitis that overcomes the disadvantages of known methods and materials.

In the case of soft tissue repair, surgical meshes and scaffolds are widely used for various reconstructions, strengthening tissues, providing support for internal organs, and treating surgical or traumatic wounds. They are usually made of inert materials and polymers such as Teflon®, polypropylene, polyglycolic acid, polyester, polyglactin 910, etc., although a titanium mesh has been used in some spinal surgeries. Alternatively, the use of tissue based materials such as acellular dermal matrix (ADM) from human and animal derived dermis is also becoming more popular.

Surgical mesh devices are typically biocompatible and can be made from bioresorbable and/or non-bioresorbable material. For example, Teflon®, polypropylene and polyester are biocompatible and non-bioresorbable while polyglycolic acid and polyglactin 910 are biocompatible and bioresorbable. ADM is processed by removing the cells and epidermis, if applicable, from the donor tissue and leaving only natural biologic components. The most common tissue based materials are human, porcine or equine derived dermal matrix.

The use of ADM has advantages against the common surgical mesh devices by lowering the rate of infection; however despite its low overall complication rate, the procedure is not without risk since ADM can generate a host inflammatory reaction and sometimes present infection. Also, it is very important to note that the properties of ADM are limited to the properties of the tissue that is harvested which can result in variability.

Furthermore, most biomaterials available today do not possess the mechanical integrity of high load demand applications (e.g., bone, ligaments, tendons, muscle) or the appropriate biological functionality; most biomaterials either degrade too rapidly (e.g., collagen, PLA, PGA, or related copolymers) or are non-degradable (e.g., polyesters, metal), where in either case, functional autologous tissue fails to develop and the patient suffers disability. In certain instances a biomaterial may misdirect tissue differentiation and development (e.g., spontaneous bone formation, tumors) because it lacks biocompatibility with surrounding cells and tissue. As well, a biomaterial that fails to degrade typically is associated with chronic inflammation, where such a response is actually detrimental to (i.e., weakens) surrounding tissue.

Thus, a need exists for an alternate or improved treatment for a foot disease, condition or ailment such as plantar fasciitis.

### SUMMARY

The present invention provider an alternate or improved treatment for a foot disease, condition or ailment such as plantar fasciitis and overcomes the disadvantages associated with known treatment methods. In one embodiment the present invention is directed to a mesh or fabric for treating plantar fasciitis. Preferably the mesh or fabric comprises, consists of or consists essentially of silk, such as a silk mesh derived from a natural source such as a silk worm. More preferably, the silk mesh comprises sericin-extracted silk fibroin fibers and a corticosteroid coated or embedded in the silk mesh. The silk mesh can be use to treat plantar fasciitis by applying a biocompatible silk mesh over a plantar aspect of a foot to treat or support plantar fascia tissue. The method can optionally further comprises impregnating the silk mesh with a corticosteroid.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DRAWINGS

The present invention will become more fully understood from the detailed description and the accompanying drawings, which are not necessarily to scale, wherein:

FIGS. 1A and 1B are bottom views of a human foot.

FIG. 1C is a side view of the foot of FIGS. 1A and 1B with a raised heel.

FIG. 1D is a side view of the foot of FIGS. 1A and 1B without a raised heel.

FIG. 2 is an enlarged side view of a foot.

FIG. 3 is another bottom and side view of a foot.

FIG. 4 is a bottom view of a foot with typical area of pain depicted.

FIG. 5 illustrates a cross-section of a silk mesh having an open knit structure suitable for use in accordance with the present invention.

### DESCRIPTION

The following detailed description of the embodiment(s) is merely exemplary in nature and is in no way intended to limit the invention, its application, or uses.

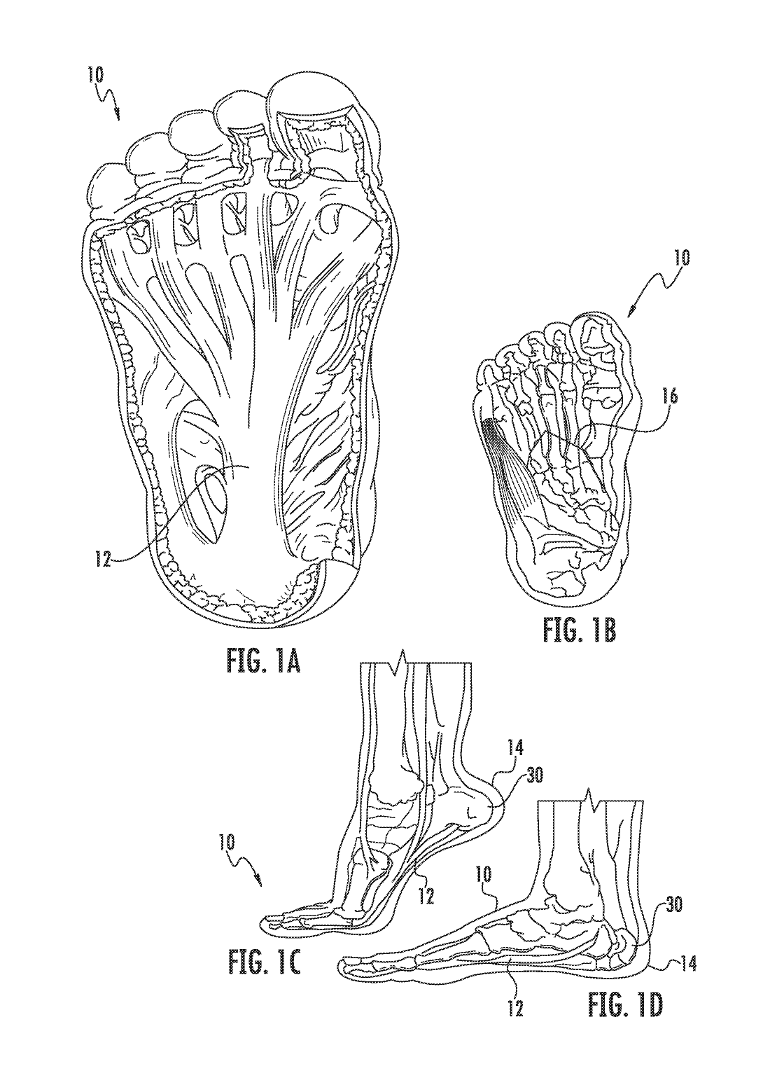

The present invention provides a method of treatment for plantar fasciitis which is a medical condition of the foot and for a surgical mesh foot implant for treatment or repair. Referring to the figures, FIG. 1A and FIG. 1B are bottom views of a foot 10. As shown in FIG. 1A, the plantar fascia 12 is a fibrous ligament at the bottom of the foot 10. As shown in FIG. 1B, nerves 16 run throughout the bottom of the foot.

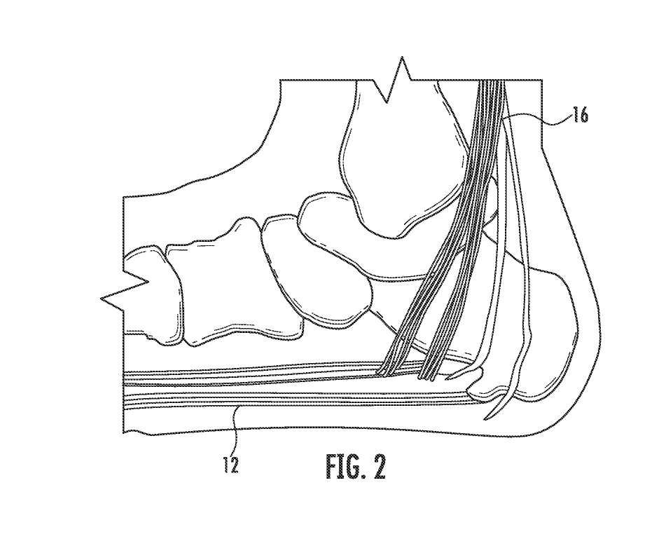

FIG. 1C represents a side view of foot 10 with a raised heel 14 in which the heel bone (calcaneus) 30 and plantar fascia 12 are shown. FIG. 1D represents a side view of a foot 10 without a raised heel 14 in which the heel bone (calcaneus) 30 and plantar fascia 12 are shown. FIG. 2 is an enlarged side view of a foot illustrating the plantar fascia 12 and nerves 16.

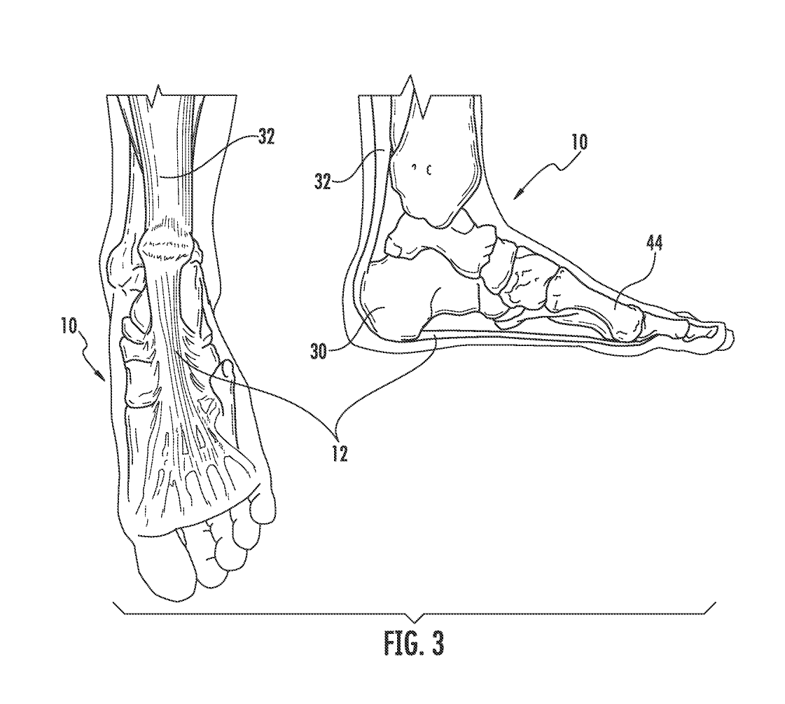

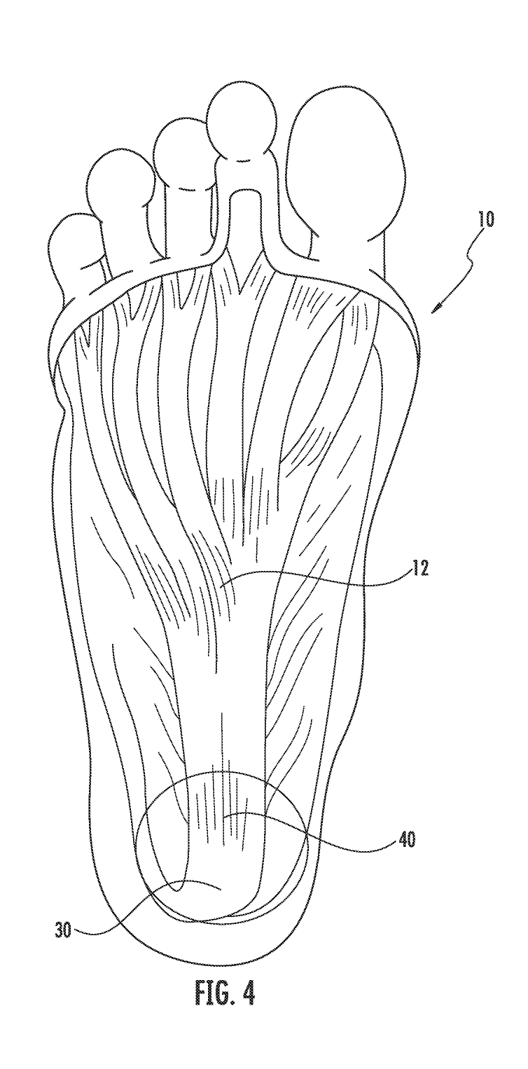

As shown in FIG. 3, the plantar fascia 12 of foot 10 is a thick fibrous band of connective tissue originating on the bottom surface of the calcaneus (heel bone) 30 and extending along the sole of the foot towards the heads of the metatarsal bones 44. The plantar fascia 12 supports the arch of the foot 10 and acts as a spring when the foot bears weight. The Achilles tendon 32 is also shown in FIG. 3. A representative area of inflammation 40 of the plantar fascia is shown in FIG. 4. The inflammation can cause heel pain which is a major source of discomfort.

The method of the present invention generally comprises obtaining a surgical mesh and applying the surgical mesh over the plantar aspect of the foot. The term “plantar aspect” refers to the bottom of the foot as opposed to the dorsum of the foot (which is the top part of the foot). More particularly, the mesh is to be applied over the plantar fascia which is the fibrous ligament at the bottom of the foot.

Once such method of application is by insertion of the surgical mesh into the foot. Insertion may occur by a percutaneous infiltration of the mesh into the foot in order to provide structural support, thereby reducing excessive tensile strain for treatment and prevention of further flare ups. The term “percutaneous” generally refers to any medical procedure where access to inner organs or tissue is done via needle-puncture of the skin, rather than by using an “open” approach where inner organs or tissue are exposed such as with the use of a scalpel. Other possible minimally invasive methods of application include, but are not limited to, arthroscopy and endoscopy.

The mesh is preferably a surgical, multi-filament, bioengineered mesh. The mesh is mechanically strong, biocompatible, and long-term bioresorbable. The mesh is a sterile mesh. It is typically used as a single use mesh and is supplied in a variety of shapes and sizes ready for use in open or laparoscopic procedures. The mesh is flexible and tear resistant with the ability to be cut in any direction. The mesh provides immediate physical and mechanical stabilization of a tissue defect through the strength and porous, scaffold-like construction of the mesh. The mesh acts as a transitory scaffold for soft tissue support and repair to reinforce deficiencies where weakness or voids exist that require the addition of material to obtain the desired surgical outcome.

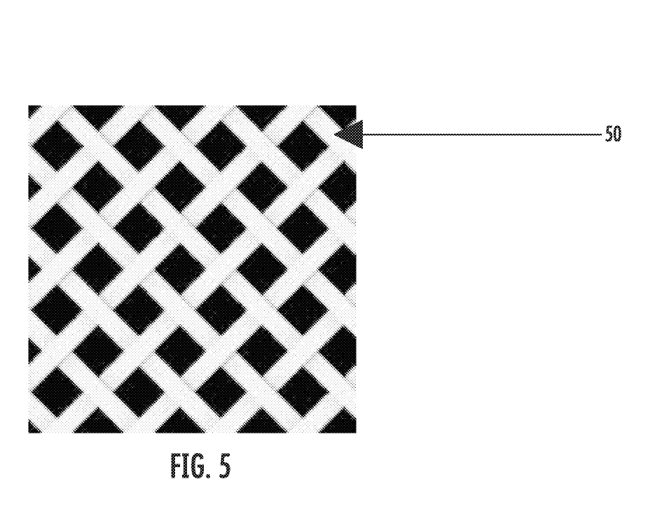

A mesh suitable for use in the method of the present invention is a silk mesh. FIG. 5 illustrates a cross-section of a silk mesh 50 having an open knit structure suitable for use in accordance with the present invention. An example of such a mesh is SeriScaffold™ silk mesh of Allergan Medical. Silk offers new clinical options for the design of a new class of medical devices, scaffolds and matrices. Silk has been shown to have the highest strength of any natural fiber, and rivals the mechanical properties of synthetic high performance fibers. Silks are also stable at high physiological temperatures and in a wide range of pH, and are insoluble in most aqueous and organic solvents. Silk is a protein, rather than a synthetic polymer, and degradation products (e.g., peptides, amino acids) are biocompatible. Silk is non-mammalian derived and carries far less bioburden than other comparable natural biomaterials (e.g., bovine or porcine derived collagen).

Silk, as the term is generally known in the art, means a filamentous fiber product secreted by an organism such as a silkworm or spider. Silks produced from insects, namely (i) Bombyx mori silkworms, and (ii) the glands of spiders, typically Nephilia clavipes, are the most often studied forms of the material; however, hundreds to thousands of natural variants of silk exist in nature. Fibroin is produced and secreted by a silkworm's two silk glands. As fibroin leaves the glands, it is coated with sericin, a glue-like substance. However, spider silk is valued (and differentiated from silkworm silk) as it is produced as a single filament lacking any immunogenic contaminates, such as sericin.

Unfortunately, spider silk cannot be mass produced due to the inability to domesticate spiders; however, spider silk, as well as other silks can be cloned and recombinantly produced, but with extremely varying results. Often, these processes introduce bioburdens, are costly, cannot yield material in significant quantities, result in highly variable material properties, and are neither tightly controlled nor reproducible.

The Bombyx mori specie of silkworm produces a silk fiber (known as a “bave”) and uses the fiber to build its cocoon. The bave, as produced, includes two fibroin filaments or “broins”, which are surrounded with a coating of gum, known as sericin—the silk fibroin filament possesses significant mechanical integrity. When silk fibers are harvested for producing yarns or textiles, including sutures, a plurality of fibers can be aligned together, and the sericin is partially dissolved and then resolidified to create a larger silk fiber structure having more than two broins mutually embedded in a sericin coating.

As used herein, “fibroin” includes silkworm fibroin (i.e. from Bombyx mori) and fibroin-like fibers obtained from spiders (i.e. from Nephila clavipes). Alternatively, silk protein suitable for use in the present invention can be obtained from a solution containing a genetically engineered silk, such as from bacteria, yeast, mammalian cells, transgenic animals or transgenic plants. See, for example, WO 97/08315 and U.S. Pat. No. 5,245,012.

Silkworm silk fibers, traditionally available on the commercial market for textile and suture applications are often “degummed” and consist of multiple broins plied together to form a larger single multi-filament fiber. Degumming here refers to the loosening of the sericin coat surrounding the two broins through washing or extraction in hot soapy water. Such loosening allows for the plying of broins to create larger multifilament single fibers. However, complete extraction is often neither attained nor desired. Degummed silk often contains or is recoated with sericin and/or sericin impurities are introduced during plying in order to congeal the multifilament single fiber. The sericin coat protects the frail fibroin filaments (only ˜5 microns in diameter) from fraying during traditional textile applications where high-through-put processing is required. Therefore, degummed silk, unless explicitly stated as sericin-free, typically contain 10-26% (by weight) sericin.

Sericin is antigenic and elicits a strong immune, allergic or hyper-T-cell type (versus the normal mild “foreign body” response) response. Sericin is removed (washed/extracted) from silk fibroin. The sericin-extracted silk fibroin fibers of the present invention are not dissolved and reconstituted.

When typically referring to “silk” in the literature, it is inferred that the remarks are focused to the naturally-occurring and only available “silk” (i.e., sericin-coated fibroin fibers) which have been used for centuries in textiles and medicine. Medical grade silkworm silk is traditionally used in only two forms: (i) as virgin silk suture, where the sericin has not been removed, and (ii) the traditional more popular silk suture, or commonly referred to as black braided silk suture, where the sericin has been completely removed, but replaced with a wax or silicone coating to provide a barrier between the silk fibroin and the body tissue and cells. Presently, the only medical application for which silk is still used is in suture ligation, particularly because silk is still valued for it mechanical properties in surgery (e.g., knot strength and handleability).

A preferred silk mesh suitable for use in the method of the present invention is a silk mesh described in co-owned and co-pending, U.S. patent application Ser. No. 12/680,404, filed Mar. 26, 2010, hereby incorporated by reference herein in its entirety. Advantageously, the open structure of such a silk mesh allows tissue in-growth while the mesh bioresorbs at a rate which allows for a smooth transfer of mechanical properties to the new tissue from the silk scaffold. FIG. 5 illustrates a silk mesh having an open knit structure. Furthermore, the silk mesh preferably employs a knit pattern that substantially prevents unraveling, especially when the mesh device is cut. In particular, embodiments may preserve the stability of the mesh device by employing a knit pattern that takes advantage of variations in tension between at least two yarns laid in a knit direction. For example, a first yarn and a second yarn may be laid in a knit direction to form “nodes” for a mesh device. The knit direction for the at least two yarns, for example, may be vertical during warp knitting or horizontal during weft knitting. The nodes of a mesh device, also known as intermesh loops, refer to intersections in the mesh device where the two yarns form a loop around a knitting needle. In some embodiments, the first yarn is applied to include greater slack than the second yarn, so that, when a load is applied to the mesh device, the first yarn is under a lower tension than the second device. A load that places the at least two yarns under tension may result, for example, when the mesh device is sutured or if there is pulling on the mesh device. The slack in the first yarn causes the first yarn to be effectively larger in diameter than the second yarn, so that the first yarn experiences greater frictional contact with the second yarn at a node and cannot move, or is “locked,” relative to the second yarn. Accordingly, this particular knit design may be referred to as a “node-lock” design.

In general, such node-lock designs employ at least two yarns under different tensions, where a higher tension yarn restricts a lower tension yarn at the mesh nodes. The at least two yarns thus differentially engage each other in a defined pattern to form a plurality of interconnections at each of which the yarns lockingly engage. To achieve variations in tension between yarns, other node-lock designs may vary the yarn diameter, the yarn materials, the yarn elastic properties, and/or the knit pattern such that the yarns are differentially engaged. For example, the knit pattern described previously applies yarns in varying lengths to create slack in some yarns so that they experience less tension. Because the lower tension yarn is restricted by the higher tension yarn, node-lock designs substantially prevent unraveling of the mesh or disengagement of the yarns from each other when tension is applied to the fabric when the mesh is cut. As such, the embodiments allow the mesh device to be cut to any shape or size while maintaining the stability of the mesh device. In addition, node-lock designs provide a stability that makes it easy to pass the mesh device through a cannula for laparoscopic or arthroscopic surgeries or insertions without damaging the material.

In another aspect of the method of the present invention, the method further comprises impregnating a silk mesh with a low dose corticosteroid for even and consistent application over the plantar aspect of the foot, for inflammatory relief while improving structural integrity to the ligaments of the foot. Suitable corticosteroids for use in the present invention include, but are not limited to, long-acting corticosteroids such as dexamethasone or betamethasone preparations. The corticosteroids are typically in low doses such as <10 mg. The mesh provides a medium to absorb corticosteroid and to distribute it in a time released fashion for long term relief while preserving structural integrity of the supportive ligaments of the plantar aspect of the foot. Another benefit of the silk mesh of the present invention includes improvement of gait by increasing dorsiflexion at the ankle.

Therefore, the present invention provides a silk-based implantable device that is biocompatible and promotes ingrowth of cells, while providing adequate support to the surrounding fascia and tissue.

### EXAMPLES

### Example 1

Plantar fasciitis of a human foot can be treated by placing a silk mesh over or by applying a silk mesh (such as SeriScaffold™ mesh) to the sole of the foot (plantar aspect of foot). The mesh is preferably impregnated with or coated low dose corticosteroid such as betamethasone in a total amount of less than about 10 mg. This can be achieved by bathing the scaffold mesh in a suitable corticosteroid solution.

### Example 2

A 40-year old healthy female can present in acute pain and exhibiting a limp. She can state that she had been having arch and heel pain of the right foot over the past month. She indicates that she recently participated in a track and field race. She states that following her race, she felt excruciating acute pain in the base of her foot the next day that made her unable to walk. She states that she has been resting at home and has taken over the counter NSAIDS for relief. Clinical evaluation of the foot reveals an extremely tender plantar fascia with localized bruising or ecchymosis. Pain is palpable along the entire course of the plantar fascia and more pronounced along the central arch. The patient is to be sent for magnetic resonance imaging (MRI) confirmation to rule out plantar fascial rupture. Pain is palpable along the entire course of the plantar fascia and more pronounced along the central arch. Diagnosis is plantar fasciitis and proposed treatment.

### Example 3

A patient can be a 22 year old female college student complaining of bilateral sharp, stabbing foot pain increasing in severity in the last three weeks. The patient can indicate that she recently took a trip to New York where she walked the city for one week. Upon her return, she began experiencing occasional sharp pain on the soles of her feet. She states that the symptoms have progressed in intensity and frequency. She currently has the most severe symptoms upon waking, and in the shower in the morning. The pain is severe enough that it causes her to “try to walk without putting my feet on the ground”. Relief position is lying on her back with a pillow under her Achilles tendons, allowing her feet to hang in plantar flexion.

Examination can reveal that plantar fascia of both feet is tight and very tender to the touch. Tight and tender bilateral calf muscles (gastrocnemius and soleus). Tender calcaneous on lateral margins. No significant tenderness is seen at the medial tubercle of the calcaneous. Joint dysfunction of the metatarsophalangeal joints and the calcaenocuneiform joints. The tibialis anterior is weak bilaterally. Orthopedic testing for a sprain or strain, nerve irritation or circulatory problem is negative. Muscle testing of the calf is within normal limits. Diagnosis is plantar fasciitis and proposed treatment is with application of silk scaffold by method of the present invention.

### Example 4

A patient can be a 34 year old avid runner, who is relatively healthy. However, after having her third child, she can report having a harder time trying to work out and has gained weight of 30 pounds since delivery. She can state that she cannot get on her treadmill to work out because she develops sever pain on the soles of her feet 10 minutes into her workout. She finds relief by elevating her legs and applying ice, but begins to feel symptoms return if she walks for long distances. Patient has a significant history for plantar fasciitis and has been treated with relief with cortiocosteoid injections but is concerned because of the risk of plantar fascia rupture following her doctor's recommendation. Upon clinical evaluation, pain is palpable along course of plantar fascia with 4+ pain along medial margins. Diagnosis is plantar fasciitis and proposed treatment is with application of silk scaffold by method of the present invention.

In the Examples above each patient can be treated specifically by: first preparing a corticosteroid solution to assist treatment of plantar fasciitis. The patient's foot is examined using diagnostic x rays and/or ultrasound prior to injection to rule-out presence of a tumor. A 25-27 gauge needle with a length of about 1.5 inches is used attached to a syringe with a capacity of about 5 ml. 1 ml of betamethasone solution (Celestone) at 6 mg/ml is used. A silk based mesh (about 6″ by 6″) such as SeriScaffold mesh is bathed in the betamethasone solution for about thirty minutes at 20 degrees C. to prepare the silk mesh for an endoscopic insertion procedure. The patient is then positioned in the lateral decubitus position with the affected foot down noting the foot landmarks: distal longitudinal crease at medial sole, proximal base of longitudinal arch, level of medial process of calcaneal tuberosity , and with soft tissue slightly distal to calcaneus. The physician identifies the point of maximal tenderness and swelling and the needle insertion site on the foot is marked based on the above noted foot landmarks . The needle is inserted at the medial foot landmark and the silk based mesh is dislodged to ensure placement.

### Example 5

### Use of a Silk Mesh in the Foot

42 year old female patient presents with tenderness on the soles of her feet which gets worse as the day goes on. This pain has started over the last 2 weeks and is making it difficult for her to walk. She has tried taking over the counter anti-inflammatories, analgesics and has applied ice over the affected area with little relief She has no significant past medical history and is currently not taking any medications. Physical examination reveals pain (3+) and tenderness (4+) on palpation of medial plantar calcaneal region and extends throughout the soles of her feet. Her feet are warm to the touch. A diagnosis of plantar fasciitisis made based on history and physical exam. Additionally, the patient can be diagnosed with plantar fasciitis based upon pain felt by the patient on the underside of the heel increasing in intensity throughout the course of the day. The patient can also have difficulty bending the foot so that the toes are brought toward the shin (decreased dorsiflexion of the ankle). Additionally, the patient is a runner and experiences knee pain.

Prior to introduction, the appropriate mesh size is chosen to ensure coverage over the plantar fascia tissue. Preferably, SeriScaffold is used as it has a lattice structure and is first bathed in a betamethasone solution.

The patient's plantar fascia region is first established using inssflation with carbon dioxide. A temporary colored vicryl stay suture is placed around the middle of the rolled mesh to keep it rolled tightly. A long suture is placed at the edge of the ball of the patient's feet on each side and tied. The rolled mesh is placed through a trocar instrument. Once the mesh enters the foot where the plantar fascia is located (plantar fascia located superficially), the rolled mesh is placed through a trochar and the mesh is unfolded. On completion of the procedure, the mesh is centered and security of the mesh is verified and the cavity is deflated. The expected treatment results are relief as betamethasone has a prompt onset of action and a is known to have a longer acting duration of effect as an anti-inflammatory medication. The SeriScaffold mesh acts as a delivery method for sustained relief to the plantar fascia region.

It will therefore be readily understood by those persons skilled in the art that the present invention is susceptible of broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

What is claimed is:

1. A method of treating plantar fasciitis, the method comprising:
applying a biocompatible silk mesh over a plantar aspect of a foot to thereby treat plantar fasciitis.

2. The method according to claim 1, wherein the applying step is carried out by insertion or implantation of the silk mesh into the plantar aspect of the foot.

3. The method according to claim 1, wherein the plantar aspect of the foot includes plantar fascia tissue.

4. The method according to claim 1, further comprising the step of impregnating a silk mesh with a corticosteroid.

5. The method according to claim 4, wherein the corticosteroid is dexamethasone or betamethasone.

6. The method according to claim 4, wherein the corticosteroid is present at a low dosage.

7. The method according to claim 1, wherein the silk mesh comprises sericin-extracted silk fibroin fibers.

8. The method according to claim 7, wherein the sericin-extracted silk fibroin fibers are not dissolved and reconstituted.

9. The method according to claim 1, wherein the silk is from a Bombyx mori silkworm.

10. The method according to claim 1, wherein the silk mesh has an open knit pattern.

11. The method according to claim 10, wherein the knit pattern substantially prevents unraveling upon cutting of the mesh.

12. The method according to claim 10, wherein the mesh has a knit pattern having at least two yarns of varying tensions laid in a knit direction.

13. A silk mesh for treatment of plantar fasciitis, the silk mesh comprising sericin-extracted silk fibroin fibers and a corticosteroid.

14. The silk mesh according to claim 13, wherein the corticosteroid is a low dose corticosteroid.

15. The silk mesh according to claim 13, wherein the silk mesh comprises sericin-extracted silk fibroin fibers.

16. The silk mesh according to claim 13, wherein the sericin-extracted silk fibroin fibers are not dissolved and reconstituted.

17. The silk mesh according to claim 13, wherein the silk is from a Bombyx mori silkworm.

18. The silk mesh according to claim 13, wherein the silk mesh has an open knit pattern.

19. The silk mesh according to claim 18, wherein the knit pattern substantially prevents unraveling upon cutting of the mesh.

20. The silk mesh according to claim 18, wherein the mesh has a knit pattern having at least two yarns of varying tensions laid in a knit direction.
